(19) United States
(12) Patent Application Publication
(10) Pub. No.: US 2010/0249655 A1
(43) Pub. Date: Sep. 30, 2010

(54) Tip-Shapeable Guidewire

(75) Inventor: Ryan R. Lemon, Sandy, UT (US)

Correspondence Address: Rutan & Tucker, LLP., 611 ANTON BLVD, SUITE 1400, COSTA MESA, CA, 92626, US

(73) Assignee: C. R. Bard, Inc., Murray Hill, NJ (US)

(21) Appl. No.: 12/749,359

(22) Filed: Mar. 29, 2010

Related U.S. Application Data
(60) Provisional application No. 61/164,845, filed on Mar. 30, 2009.

(51) Int. Cl. A61M 25/09; F28D 21/00

(52) U.S. Cl. 600/585; 432/9

(57) ABSTRACT

A guidewire for partial placement within a body of a patient is disclosed. The guidewire is employed to assist in the insertion of a medical device into the body, such as the placement of a catheter into the patient's vasculature. In one embodiment, the guidewire defines an elongate body that includes a distal segment. The distal segment includes a shape memory material that enables at least a portion of the distal segment to be deformed by a user prior to placement of the guidewire in the body of the patient. The shape memory material enables the guidewire to maintain the deformation of the distal segment portion after being deformed by the user.

### CROSS-REFERENCE TO RELATED APPLICATIONS

This application claims the benefit of U.S. Provisional Patent Application No. 61/164,845, filed Mar. 30, 2009, and entitled “Tip Shapeable Guidewire,” which is incorporated herein by reference in its entirety.

### BRIEF SUMMARY

Briefly summarized, embodiments of the present invention are directed to a guidewire for partial placement within a body of a patient. The guidewire is employed to assist in the insertion of a medical device into the body, such as the placement of a catheter into the patient's vasculature.

In one embodiment, the guidewire defines an elongate body that includes a distal segment. The distal segment includes a shape memory material that enables at least a portion of the distal segment to be deformed by a user prior to placement of the guidewire in the body of the patient. In one embodiment, the shape memory material includes a nickel-titanium alloy that is heat treated as to impart malleability to the distal segment. The shape memory material enables the guidewire to maintain the deformation of the distal segment portion after being deformed by the user. In one embodiment, more proximal portions of the guidewire also include a shape memory material and remain untreated by a heat treating process such that the proximal portions are kink-resistant.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A description of the embodiments of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIGS. 1A and 1B are perspective and cross-sectional views, respectively, of a guidewire configured in accordance with one example embodiment of the present invention;

FIG. 2 is a cross-sectional view of the guidewire of FIGS. 1A and 1B including a deformable portion thereof in one possible bent configuration;

FIG. 3 is a cross-sectional view of the guidewire of FIGS. 1A and 1B including a deformable portion thereof in another possible bent configuration;

FIG. 4 is a cross-sectional view of a distal segment a guidewire in accordance with one embodiment; and

FIG. 5 is a cross-sectional view of a distal segment of a guidewire in accordance with another embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

For clarity it is to be understood that the word “proximal” refers to a direction relatively closer to a clinician using the device to be described herein, while the word “distal” refers to a direction relatively further from the clinician. For example, the end of a guidewire placed within the body of a patient is considered a distal end of the guidewire, while the guidewire end remaining outside the body is a proximal end of the guidewire. Also, the words “including,” “has,” and “having,” as used herein, including the claims, shall have the same meaning as the word “comprising.”

As used herein, “shape memory material” is understood to include a material that retains a deformed shape after deformation from an original shape, but can recover its original shape when subjected to a suitable restorative action. Non-limiting examples of shape memory materials include shape memory alloys, shape memory polymers, and ferromagnetic shape memory alloys.

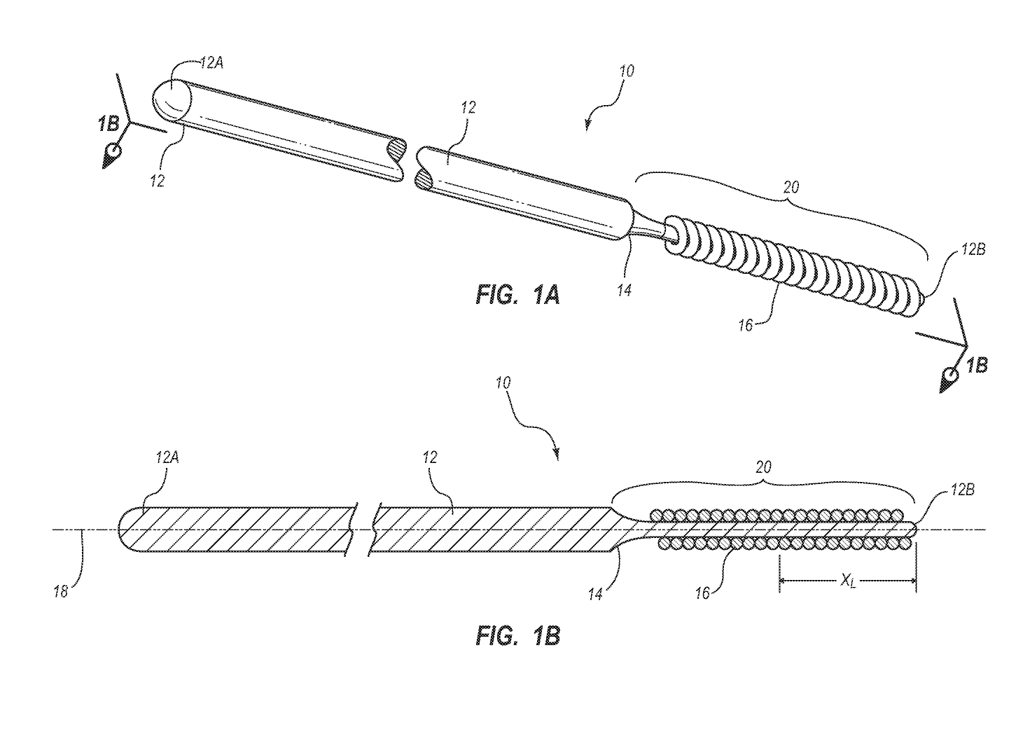

FIGS. 1A-5 depict various features of embodiments of the present invention, which are generally directed to a guidewire for use in assisting with the placement of medical devices into a body of a patient. In one embodiment, the guidewire is employed to assist with the placement of a catheter into a vasculature of the patient, for instance.

In one embodiment, the guidewire includes a deformable distal portion that enables a clinician or other user to manipulate the deformable portion into a desired shape. In one embodiment the deformable portion includes a memory shape material, such as a nickel-titanium alloy for example, that enables the deformable portion to retain the deformed shape after the deforming force used to shape the portion is removed. Thus, an initially straight guidewire distal segment can be modified in any one of a variety of possible shapes as may be desired by the clinician, in preparation for inserting the guidewire into a vasculature of a patient.

Reference is first made to FIGS. 1A and 1B in describing various details regarding a guidewire, generally designated at 10, configured in accordance with one embodiment. As shown, the guidewire 10 includes an elongate body 12 defining a proximal end 12A, a distal end 12B, and a longitudinal axis 18.

A reduced diameter portion 14 is defined toward the distal end 12B of the guidewire body 12 and defines a transition from a diameter defined by more proximal portions of the guidewire to a reduced diameter distal segment 20 of the guidewire body adjacent the distal end thereof.

Optionally, an atraumatic coil 16 is disposed about the reduced diameter distal segment 20 to enable atraumatic advancement of the guidewire 10 through a vasculature of a patient in connection with the initial placement or exchange placement of a catheter, for instance, or other medical device configured for insertion into a body of a patient. The coil 16 may include stainless steel, platinum, gold-tungsten, or other suitable material. It is appreciated that the length, diameter, and overall configuration of the guidewire body, including the distal segment, can vary from what is explicitly shown herein while still benefiting from the principles disclosed in this and other embodiments.

In the present embodiment, the distal portion 20 of the guidewire body 12 includes a deformable portion that is shapeable, or deformable, from its linear configuration shown in FIGS. 1A and 1B, when subjected to a deforming force. Moreover, the distal portion 20 is configured to maintain the deformed configuration after the deforming force has been removed. Such deformability is useful, for instance, in situations where a clinician desires to manually deform a portion of the guidewire distal segment 20 into a shape other than a linear configuration before inserting the guidewire into the patient's vasculature. FIG. 1B shows that in one embodiment a portion of the distal segment 20 of length X_{L} is deformable. In other embodiments, of course, more or less of the distal segment can be configured for deformation.

The guidewire 10 includes a material that enables deformation of a portion of the distal segment 20 as described above. In particular, in the present embodiment, the guidewire distal portion 20 includes a shape memory material such as a nickel-titanium alloy, commonly known as nitinol. The inclusion of nitinol in the distal segment 20 enables the distal segment to be deformed into a shaped configuration as desired by the clinician, then to maintain the shape for later insertion of the guidewire into the body. In one embodiment, the distal segment 20 includes about 50.8 atomic percent nickel and about 49.2 percent atomic percent titanium, by volume, though it is appreciated that in other embodiments other relative concentrations can be employed.

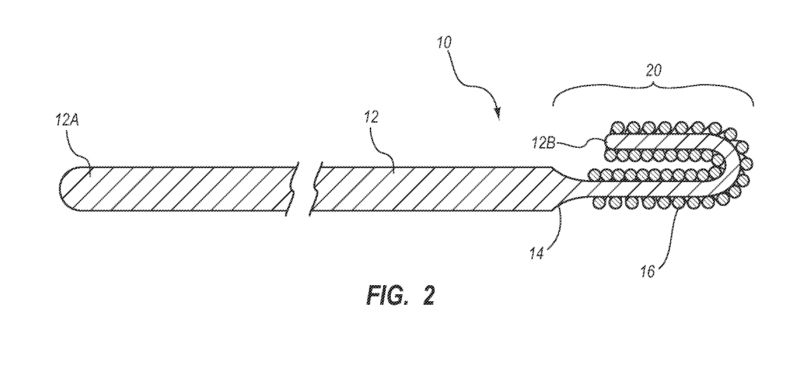

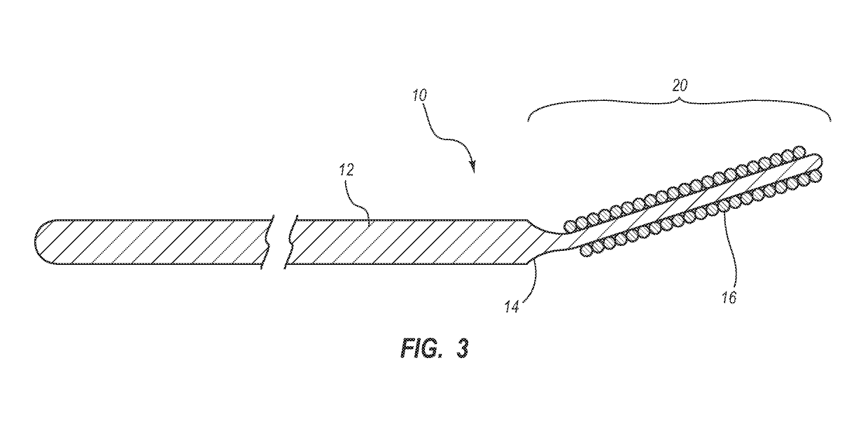

FIGS. 2 and 3 show non-limiting examples of how a clinician can deform the distal segment 20 to a shaped configuration in preparation for advancing the guidewire 10 into the patient vasculature. FIG. 2, for example, depicts the distal segment 20 after deformation by a deformation force, such as manual manipulation, into a “J-tip” configuration. FIG. 3 depicts the distal segment 20 deformed into a modified J-tip configuration, wherein the entire distal segment 20 is bent, so as to deviate from the longitudinal axis 18 (FIG. 1B). Of course, various other tip deformation configurations are contemplated. Note that the shape of the distal segment 20 does not change after removal of the deformation force in the present embodiment. Note also that in one embodiment, the deformable portion may include only a portion of the distal segment. Note further that, in one embodiment, the guidewire distal segment can be pre-deformed into a shaped configuration such that no further deformation by the user is necessary.

In one embodiment, the guidewire body 12 includes nitinol and configured to exhibit superelastic characteristics. The guidewire distal segment 20 of the guidewire body 12 is annealed, or heat-treated, so as to remove superelastic characteristics therefrom and instead impart deformable characteristics to the distal segment. In the present embodiment, the heat treating process is performed while the distal segment 20 is positioned in an un-bent configuration with respect to the longitudinal axis 18 of the guidewire 10. During the heat-treating process, the distal segment 20 is heated to a predetermined temperature and then cooled in a predetermined manner to modify the molecular structure of the material. Heat-treatment of the nitinol distal segment 20 in this manner causes the distal segment to lose its superelastic characteristics and become malleable, thus suitable for deformation, while the remaining proximal portion of the guidewire body 12 retains its kink-resistant, superelastic characteristics.

In one embodiment, the distal segment 20 can be heat-treated in a conventional oven, an IR oven, by laser, or by any other suitable method. Again, it is appreciated that the portion of the distal segment or guidewire that is treated in this manner can vary according to need or desire, and that other portions of the guidewire can undergo such a heat treating process. Of course, other stages in the formation of the guidewire according to one embodiment include reducing the diameter of the distal segment and adding an atraumatic coil thereto via UV or epoxy adhesive, soldering, etc. These stages can occur before or after heat treatment.

After suitable heat-treatment of the distal segment 20 as described above, the untreated proximal portion of the nitinol guidewire body 12 retains its superelastic properties so as to offer kink resistance to the guidewire 10. In contrast, the heat-treated nitinol distal segment 20 is malleable so as to be selectively deformed by a clinician, manually or via mechanical assistance for example, in preparation for advancement of the guidewire 10 into the vasculature of the patient during a catheter placement or other procedure. Optionally, a deformable shape memory guidewire body can be manufactured, then the portion of the body proximal to the distal segment can be treated so as to impart thereto superelastic characteristics, in one embodiment.

In one embodiment where at least the distal segment 20 includes a shape memory material such as nitinol, the distal segment is heat-treated during manufacture as described above in order to impart the desired deformable characteristics thereto. Later, a clinician can deform all or a portion of the guidewire distal segment 20 to a desired shape. Once the distal segment 20 is suitably shaped, the guidewire 10 can be inserted into the patient's body in accordance with typical procedures. Again, the length of the heat treated distal segment relative to the length of the guidewire can vary from what is depicted in the accompanying drawings. Also, it is appreciated that the guidewire can be shaped and re-shaped multiple times, if desired.

It is appreciated that the relative portion of the guidewire including a shape memory material can vary. In one embodiment, for instance, the entire guidewire body 12 includes a shape memory material. In another embodiment, the distal segment 20 includes a shape memory material while more proximal portions of the guidewire include another material, such as stainless steel, for instance. Note that in addition to nitinol, other shape memory materials can be employed, such as other shape memory alloys, shape memory polymers, and ferromagnetic shape memory alloys, and for instance.

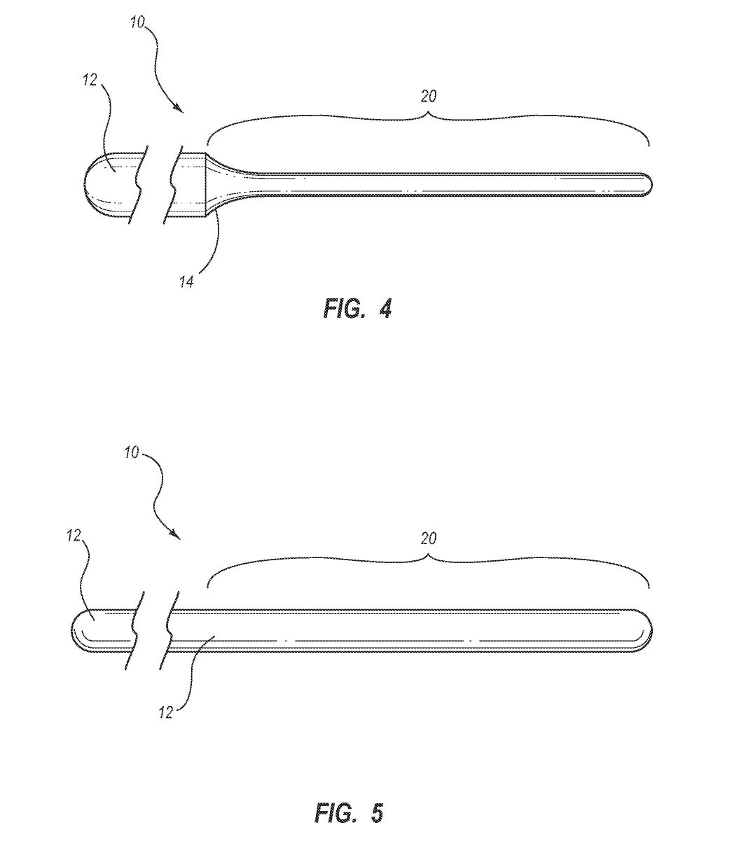

FIGS. 4 and 5 show different configurations of the guidewire distal segment 20, according to additional embodiments. FIG. 4 depicts the heat-treated distal segment 20 as in previous embodiments, but without the atraumatic coil disposed thereabout. FIG. 5 depicts the distal segment 20 wherein the distal segment does not include the atraumatic coil and is not reduced in diameter with respect to more proximal portions of the guidewire 10. These configurations therefore serve as non-limiting examples of the manner in which the distal segment can be modified in accordance with embodiments of the present invention.

In embodiments where it is employed in connection with insertion of a catheter within the vasculature of a patient, the guidewire 10 is first positioned within the vasculature, and the catheter is subsequently advanced over the guidewire. In another embodiment, the guidewire can be disposed within a lumen of the catheter and both the catheter and the guidewire are simultaneously inserted into the patient's vasculature. In this latter case, the guidewire functions as a stylet. In either embodiment the guidewire/stylet as described herein assists in providing for a reduced-trauma catheter insertion procedure.

Embodiments of the present invention may be embodied in other specific forms without departing from its spirit or essential characteristics. The described embodiments are to be considered in all respects only as illustrative, not restrictive. The scope of embodiments of the invention is, therefore, indicated by the appended claims rather than by the foregoing description. All changes that come within the meaning and range of equivalency of the claims are to be embraced within their scope.

What is claimed is:

1. A guidewire for partial placement within a body of a patient, comprising:
an elongate body including a shape memory material and defining a distal segment, wherein at least a portion of the distal segment capable of being deformed prior to placement of the guidewire in the body of the patient, the distal segment remaining deformed after deformation, and wherein a portion of the guidewire proximal to the distal segment includes superelastic characteristics.

2. The guidewire as defined in claim 1, wherein an entirety of the elongate body includes a single shape memory material.

3. The guidewire as defined in claim 2, wherein an entirety of the elongate body proximal to the distal segment includes superelastic characteristics.

4. The guidewire as defined in claim 3, wherein the guidewire includes a nickel-titanium alloy.

5. The guidewire as defined in claim 1, wherein the deformation of the portion of the distal segment is performed by a user and the deformation is maintained after placement of at least the distal segment in the vasculature of the patient.

6. The guidewire as defined in claim 1, wherein the distal segment includes a reduced diameter with respect to more proximal portions of the elongate body.

7. The guidewire as defined in claim 1, wherein the guidewire is configured such that a catheter can slide over the guidewire for placement of the catheter within a vasculature of the patient.

8. A method for manufacturing a guidewire, comprising:
forming an elongate guidewire body including a shape memory material; and
heat treating a distal segment so as to impart deformability to the distal segment, wherein the distal segment maintains a deformed configuration after the distal segment is deformed by a user, and wherein the elongate body includes at least one portion proximal to the distal segment that does not maintain a deformed configuration.

9. The method for manufacturing as defined in claim 8, wherein the at least one portion proximal to the distal segment retains superelastic characteristics.

10. A method for manufacturing a guidewire, comprising:
forming an elongate guidewire body including a shape memory material; and
treating one of a distal segment of the elongate body and a portion of the elongate body proximal to the distal segment such that the distal segment includes deformability so as to maintain a deformed configuration after the distal segment is deformed by a user, and such that the portion proximal to the distal segment includes superelastic characteristics.

11. The method for manufacturing as defined in claim 10, wherein treating one of a distal segment further comprises:
heat treating the distal segment of the elongate body, the shape memory material of the distal segment including superelastic characteristics before heat treating thereof.

12. A guidewire for placement within a body of a patient, comprising:
an elongate body including a distal segment, the distal segment including a heat treated nickel-titanium shape memory alloy such that the distal segment can be deformed by a deforming force, the distal segment maintaining the deformed state after a deforming force is removed, wherein a portion of the elongate body proximal to the distal segment is not configured to maintain a shape memory deformation after deformation thereof.

13. The guidewire as defined in claim 12, wherein the deforming force is provided by a clinician prior to advancement into the body of the patient

14. The guidewire as defined in claim 12, wherein the distal segment can be shaped into a J-tip configuration.

15. The guidewire as defined in claim 12, wherein an entirety of the elongate body includes the nickel-titanium alloy.

16. The guidewire as defined in claim 12, wherein a proximal portion of the elongate body proximal to the distal segment includes stainless steel.

17. The guidewire as defined in claim 12, wherein the distal segment includes a reduced diameter with respect to more proximal portions of the elongate body.

18. The guidewire as defined in claim 17, wherein the distal segment includes an atraumatic coil.

19. A method for using a guidewire, comprising:
providing a guidewire including a distal segment, the distal segment including a shape memory alloy;
deforming at least a portion of the distal segment to a shaped configuration, the guidewire maintaining the shaped configuration after deformation, a portion of the guidewire proximal to the distal segment including superelastic characteristics; and
inserting the distal segment including the shaped configuration into a vasculature of the patient.

20. The method for using the guidewire as defined in claim 19, wherein deforming at least a portion of the distal segment further includes:
deforming at least a portion of the distal segment by a deforming force provided by a user of the guidewire.

21. The method for using a guidewire as defined in claim 19, wherein deforming at least a portion of the distal segment deviates the portion from a longitudinal axis of a proximal portion of the guidewire.
